(19) United States
(12) Patent Application Publication
(10) Pub. No.: US 2023/0405162 A1
(43) Pub. Date: Dec. 21, 2023

(54) EXTERNAL STERILIZATION OF AEROSOL-GENERATING DEVICES

(71) Applicant: Altria Client Services LLC, Richmond, VA (US)

(72) Inventor: Christopher J. SUAREZ, Richmond, VA (US)

(73) Assignee: Altria Client Services LLC, Richmond, VA (US)

(21) Appl. No.: 17/845,237

(22) Filed: Jun. 21, 2022

(51) Int. Cl. A61L 2/10; A61L 2/26; A24F 40/90

(52) U.S. Cl. CPC A61L 2/10; A61L 2/26; A24F 40/90; A61L 2202/122

(57) ABSTRACT

A sterilization system for an aerosol-generating device includes a charging station and a sterilization device. The charging station is configured to receive the aerosol-generating device. The sterilization device includes at least one UV diode. The sterilization device is configured to couple to the charging station and surround the aerosol-generating device when the aerosol-generating device has been received by the charging station. The at least one UV diode is configured to sanitize an exterior of the aerosol-generating device when the at least one UV diode is actuated.

### TECHNICAL FIELD

At least some example embodiments relate generally to external sterilization of aerosol-generating devices.

### BACKGROUND

Some electronic devices are configured to heat a plant material to a temperature that is sufficient to release constituents of the plant material while keeping the temperature below its ignition temperature so as to avoid a self-sustaining burning or a self-sustaining combustion of the plant material (i.e., in contrast to where a plant material is lit, such as lit-end cigarettes). Such devices may be characterized as generating an aerosol of constituents released by heating and may be referred to as heat-not-burn aerosol-generating devices, or heat-not-burn devices.

It is understood that heating of a plant material below its ignition temperature may, in some circumstances, produce incidental and insubstantial levels of oxidized or other thermal decomposition byproducts. However, in some embodiments, the heating in aerosol-generating devices is below the pyrolysis temperature of the plant material so as to produce an aerosol having no or insubstantial levels of thermal decomposition byproducts of the plant material. Thus, in an example embodiment, pyrolysis of the plant material does not occur during the heating and resulting production of aerosol. In other instances, there may be incidental pyrolysis, with production of oxidized or other thermal decomposition byproducts at levels that are insignificant relative to the primary constituents released by heating of the plant materials.

### BRIEF SUMMARY

New and useful systems, apparatuses, and methods for external sterilization of aerosol-generating devices are set forth in the appended claims. Illustrative embodiments are also provided to enable a person skilled in the art to make and use the claimed subject matter.

For example, in some example embodiments, a sterilization system for an aerosol-generating device can include a charging station and a sterilization device. The charging station can be configured to receive the aerosol-generating device. The sterilization device can include at least one UV diode. The sterilization device can be configured to couple to the charging station and surround the aerosol-generating device when the aerosol-generating device has been received by the charging station. The at least one UV diode can be configured to sterilize an exterior of the aerosol-generating device when the at least one UV diode is actuated.

In some example embodiments, the sterilization device can include a first end with a central portion and a periphery surrounding the central portion. The sterilization device can also include an enclosure extending from the periphery of the first end. The enclosure can be configured to couple to the charging station. In some example embodiments, the first end of the sterilization device can include a first surface with at least one UV diode and a second surface opposite the first surface. The at least one UV diode can be directed away from the second surface of the sterilization device. In some example embodiments, the at least one UV diode can include a plurality of UV diodes arranged in a circle on the first surface of the first end of the sterilization device. In other example embodiments, the at least one UV diode can include a plurality of UV diodes arranged to direct UV light to the exterior of the aerosol-generating device. In some example embodiments, the at least one UV diode includes a plurality of UV diodes on the first surface of the first end of the sterilization device directed towards the charging station such that UV light from the plurality of UV diodes contact an entire exterior of the aerosol-generating device when the aerosol-generating device is coupled to the charging station.

In some example embodiments, the enclosure can include at least one magnet configured to couple to at least one magnet of the charging station. The at least one magnet of the enclosure and the at least one magnet of the charging station can be neodymium magnets.

In some example embodiments, the at least one UV diode is actuated when the aerosol-generating device is received by the charging station and when the sterilization device is coupled to the charging station.

In some example embodiments, the at least one UV diode can be turned off if the sterilization device is decoupled from the charging station.

In some example embodiments, the charging station can include a communication screen. The communication screen can be configured to output information related to a charge status of the aerosol-generating device. The communication screen can also be configured to output information related to a sterilization status of the aerosol-generating device.

In some embodiments, the sterilization device can be communicatively coupled to the charging station. The sterilization device can be communicatively coupled to the charging station at a contact point. The contact point can be configured to be communicatively coupled to the at least one UV diode in some embodiments.

In some example embodiments, the sterilization device can include a plastic UV filter.

In some example embodiments, the at least one UV diode can include a UV-C diode.

In some example embodiments, the at least one UV diode can include a UV-C light ring diode.

In some example embodiments, the at least one UV diode can be configured to emit UV light in a wavelength range of 100 nanometers to 280 nanometers.

Also described herein is a method of sanitizing an exterior of an aerosol-generating device. In some example embodiments, the method can include coupling the aerosol-generating device to a charging station, coupling a sterilization device to the charging station such that the sterilization device surrounds the aerosol-generating device, actuating the at least one UV diode of the sterilization device, and operating the at least one UV diode for a predetermined or a desired amount of time.

In some example embodiments, the predetermined amount of time can be an amount of time to sanitize the exterior of the aerosol-generating device.

In some example embodiments, the at least one UV diode can be turned off if the sterilization device is decoupled from the charging station.

In some example embodiments, the method can further include outputting a sanitization status and a charging status on a communication screen of the charging station.

In some example embodiments, actuating the at least one UV diode of the sterilization device can include directing UV light from the at least one UV diode to the exterior of the aerosol-generating device to sanitize the exterior of the aerosol-generating device.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

The various features and advantages of the non-limiting embodiments herein may become more apparent upon review of the detailed description in conjunction with the accompanying drawings. The accompanying drawings are merely provided for illustrative purposes and should not be interpreted to limit the scope of the claims. The accompanying drawings are not to be considered as drawn to scale unless explicitly noted. For purposes of clarity, various dimensions of the drawings may have been exaggerated.

FIG. 1 is a front perspective view of a sterilization system in accordance with at least one example embodiment.

FIG. 2 is a top right, front exploded view of the sterilization system illustrated in FIG. 1.

FIG. 3 is a top right, front perspective view of the sterilization system of FIG. 1 with an aerosol-generating device housed within the sterilization system.

FIG. 4 is a top-down view of the sterilization system of FIG. 3.

FIG. 5 is a top right, front perspective view of the sterilization system of FIG. 3 in operation.

FIG. 6 is a block diagram illustrating example electrical connections between various components of the sterilization system shown in FIGS. 1-5.

FIG. 7 illustrates an example embodiment of a controller of the sterilization system shown in FIG. 6.

### DESCRIPTION OF EXAMPLE EMBODIMENTS

Some detailed example embodiments are disclosed herein. However, specific structural and functional details disclosed herein are merely representative for purposes of describing example embodiments. Example embodiments may, however, be embodied in many alternate forms and should not be construed as limited to only the example embodiments set forth herein.

Accordingly, while example embodiments are capable of various modifications and alternative forms, example embodiments thereof are shown by way of example in the drawings and will herein be described in detail. It should be understood, however, that there is no intent to limit example embodiments to the particular forms disclosed, but to the contrary, example embodiments are to cover all modifications, equivalents, and alternatives falling within the scope of example embodiments. Like numbers refer to like elements throughout the description of the figures.

It should be understood that when an element or layer is referred to as being “on,” “connected to,” “coupled to,” or “covering” another element or layer, it may be directly on, connected to, coupled to, or covering the other element or layer or intervening elements or layers may be present. In contrast, when an element is referred to as being “directly on,” “directly connected to,” or “directly coupled to” another element or layer, there are no intervening elements or layers present. As used herein, the term “and/or” includes any and all combinations of one or more of the associated listed items.

It should be understood that, although the terms first, second, third, etc. may be used herein to describe various elements, regions, layers and/or sections, these elements, regions, layers, and/or sections should not be limited by these terms. These terms are only used to distinguish one element, region, layer, or section from another region, layer, or section. Thus, a first element, component, region, layer, or section discussed below could be termed a second element, region, layer, or section without departing from the teachings of example embodiments.

Spatially relative terms (e.g., “beneath,” “below,” “lower,” “above,” “upper,” and the like) may be used herein for ease of description to describe one element or feature's relationship to another element(s) or feature(s) as illustrated in the figures. It should be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” may encompass both an orientation of above and below. The device may be otherwise oriented (rotated 90 degrees or at other orientations) and the spatially relative descriptors used herein interpreted accordingly.

The terminology used herein is for the purpose of describing various example embodiments only and is not intended to be limiting of example embodiments. As used herein, the singular forms “a,” “an,” and “the” are intended to include the plural forms as well, unless the context clearly indicates otherwise. It will be further understood that the terms “includes,” “including,” “comprises,” and/or “comprising,” specify the presence of stated features, integers, steps, operations, and/or elements, but do not preclude the presence or addition of one or more other features, integers, steps, operations, elements, and/or groups thereof.

When the terms “about” or “substantially” are used in this specification in connection with a numerical value, it is intended that the associated numerical value includes a manufacturing or operational tolerance (e.g., ±10%) around the stated numerical value. Moreover, when the terms “generally” or “substantially” are used in connection with geometric shapes, it is intended that precision of the geometric shape is not required but that latitude for the shape is within the scope of the disclosure. Furthermore, regardless of whether numerical values or shapes are modified as “about,” “generally,” or “substantially,” it will be understood that these values and shapes should be construed as including a manufacturing or operational tolerance (e.g., ±10%) around the stated numerical values or shapes.

Unless otherwise defined, all terms (including technical and scientific terms) used herein have the same meaning as commonly understood by one of ordinary skill in the art to which example embodiments belong. It will be further understood that terms, including those defined in commonly used dictionaries, should be interpreted as having a meaning that is consistent with their meaning in the context of the relevant art and will not be interpreted in an idealized or overly formal sense unless expressly so defined herein.

As used herein, “coupled” includes both removably coupled and permanently coupled. For example, when an elastic layer and a support layer are removably coupled to one another, the elastic layer and the support layer can be separated upon the application of sufficient force.

Hardware may be implemented using processing or control circuitry such as, but not limited to, one or more processors, one or more Central Processing Units (CPUs), one or more microcontrollers, one or more arithmetic logic units (ALUs), one or more digital signal processors (DSPs), one or more microcomputers, one or more field programmable gate arrays (FPGAs), one or more System-on-Chips (SoCs), one or more programmable logic units (PLUs), one or more microprocessors, one or more Application Specific Integrated Circuits (ASICs), or any other device or devices capable of responding to and executing instructions in a defined manner.

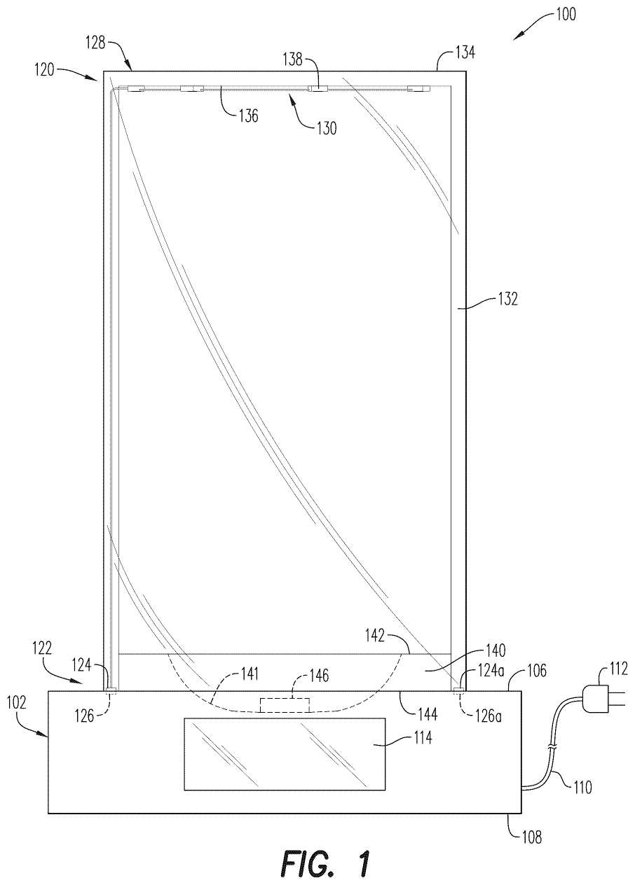

Referring to FIG. 1, a front view of a sterilization system 100 is shown. In some embodiments, the sterilization system 100 may include a charging station 102 and a sterilization device 104. The charging station 102 may have a first surface 106 and a second surface 108 opposite the first surface 106. The second surface 108 may be configured to support the sterilization system 100 in some embodiments. The first surface 106 may be configured to couple to the sterilization device 104. In some embodiments, the charging station 102 may include a charger input that may include a wire 110 and a plug 112 to couple the charging station 102 to an outlet to provide power to the sterilization system 100. In other embodiments, the charging station 102 may include batteries or another on-board power source that can provide power to the sterilization system 100 without the need for the wire 110 and the plug 112.

In some embodiments, the charging station 102 may further include a communication screen 114. In some embodiments, the communication screen 114 may be an integrated thin-film transistor (“TFT”) screen. In other example embodiments, the communication screen 114 may be an organic light emitting diode (“OLED”) or light emitting diode (“LED”) screen. The communication screen 114 may be configured for adult consumer engagement in some embodiments. The communication screen 114 may be configured to output information related to a charging status or a sterilization status of an aerosol-generating device. For example, the communication screen 114 may output a time indicating how long it will take to fully charge and/or fully sanitize the aerosol-generating device.

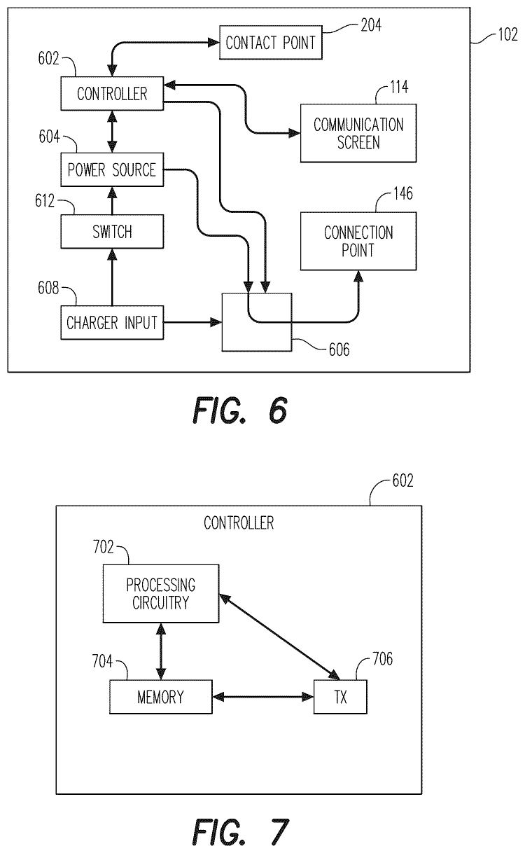

In some embodiments, the charging station 102 may include a controller or processor that may be configured to receive and output information to different components of the sterilization system 100. For example, the controller may be configured to operate the charging station 102 in a charging mode for a certain amount of time to fully charge an aerosol-generating device that is coupled to the charging station 102. The controller may further be configured to process and output information about the charging status on the communication screen 114 in some embodiments. Various example connections between the controller and the different components of the sterilization system 100 will be further described below with reference to FIG. 6.

The sterilization device 104 may have a first end 120 and a second end 122 opposite the first end 120. The second end 122 may couple to the first surface 106 of the charging station 102. In some embodiments, the second end 122 of the sterilization device 104 may include a magnet 124. The magnet 124 may couple with a base magnet 126 in the first surface 106 of the charging station 102 to removably couple the sterilization device 104 to the charging station 102. In some embodiments, the magnet 124 and the base magnet 126 may be neodymium magnets. In other embodiments, the magnet 124 and the base magnet 126 may be other magnets that may releasably couple the sterilization device 104 to the charging station 102. In some embodiments, there may be a second pair of magnets such as magnet 124a and base magnet 126a that may be disposed opposite the magnet 124 and the base magnet 126. The magnet 124a and the base magnet 126a may be similar or equivalent to the magnet 124 and the base magnet 126 in some embodiments. In other embodiments, the sterilization device 104 and the charging station 102 may include additional magnets configured to couple the sterilization device 104 to the charging station 102.

The first end 120 of the sterilization device 104 may include a peripheral portion or a periphery 128 and a central portion 130. The periphery 128 may surround the central portion 130. An enclosure 132 may extend from the periphery 128 to the second end 122 of the sterilization device 104. In some embodiments, the magnet 124 and the magnet 124a may be disposed within the enclosure 132 at the second end 122 of the sterilization device 104. In other embodiments, the magnet 124 and the magnet 124a may be coupled to the enclosure 132 by an adhesive, a weld, or another method of coupling to secure the magnet 124 and the magnet 124a to the enclosure 132.

The first end 120 may further include a first surface 134 and a second surface 136 opposite the first surface 134. When the sterilization device 104 is coupled to the charging station 102, the first surface 134 may be exposed to an ambient environment surrounding the sterilization system 100 and the second surface 136 may be isolated from the ambient environment surrounding the sterilization system 100. The second surface 136 of the sterilization device 104 may include at least one UV diode 138. The at least one UV diode 138 may be directed away from the first surface 134 of the first end 120 of the sterilization device 104. In some embodiments, the at least one UV diode 138 may include a plurality of UV diodes 138. The plurality of UV diodes 138 may be arranged in a circle in some embodiments. In other embodiments, the plurality of UV diodes 138 may be arranged in another pattern such as a square, a hexagon, or another shape. In still other embodiments, the plurality of UV diodes 138 may be arranged randomly on the second surface 136. In any of the above embodiments, the at least one UV diode 138 or the plurality of UV diodes may be arranged to direct UV light to an area defined by the enclosure 132 and an exterior of the aerosol-generating device. More specifically, UV light from the at least one UV diode 138 or the plurality of UV diodes may contact an entire exterior of the aerosol-generating device when the aerosol-generating device is coupled to the charging station 102.

In some embodiments, the at least one UV diode 138 may be a UV-C light ring diode. In other embodiments, the at least one UV diode 138 may include a UV-C diode. The at least one UV diode 138 may be configured to emit UV light in a wavelength in the range of 100 nanometers to 280 nanometers in some embodiments.

In some embodiments, the controller may be configured to operate the at least one UV diode 138. For example, the controller may be configured to operate the at least one UV diode for a predetermined or a desired amount of time in order to fully sanitize an exterior of the aerosol-generating device. The controller may further be configured to process and output information about the sanitization status on the communication screen 114 in some embodiments. Additionally, the controller may be able to receive updates from an external source such as a mobile device or a computer with updates to the desired wavelengths or time to operate the at least one UV diode 138. The controller may receive updates to operating parameters for the at least one UV diode and may apply those updates without any intervention from an adult consumer.

In some embodiments, as shown in FIGS. 1-5, the sterilization device 104 may be transparent. More specifically, the sterilization device 104 may be formed from a plastic material that is a UV filter. The sterilization device 104 may be formed from a plastic material that is a UV filter so that UV light from the at least one UV diode 138 remains within the enclosure 132 of the sterilization device 104 and does not escape the sterilization system 100. The plastic material of the sterilization device 104 may be configured to block UV-C light between 100 nanometers and 180 nanometers. In some embodiments the plastic material may be a UV filtering plexiglass acrylic sheet such as OP3/UF-5 Acrylite UV filtering (OP-3) acrylic sheet or a UV filtering museum grade plexiglass sheet such as OP2/UF-3 UV filtering OP-2 Acrylite/UF-3. In some embodiments, it may be desirable to use plexiglass plastic filters for the sterilization device 104.

In some embodiments, the charging station 102 may further include a device holder or a dock 140. The dock 140 may extend from the first surface 106 of the charging station 102 past the second end 122 of the sterilization device 104 towards the first end 120 of the sterilization device 104. The dock 140 may be configured to receive an aerosol-generating device. In some embodiments, the dock 140 may have a recess 141 extending from a first end 142 of the dock towards a second end 144 of the dock. In some embodiments the recess 141 may be spherical. In other embodiments, the recess may be a different shape but may still be configured to receive an aerosol-generating device. There may be a connection point 146 within the recess 141 of the dock 140. The connection point 146 may be configured to engage a port of an aerosol-generating device to couple the aerosol-generating device to the charging station 102. The dock 140 may be surrounded by the enclosure 132 when the sterilization device 104 is coupled to the charging station 102.

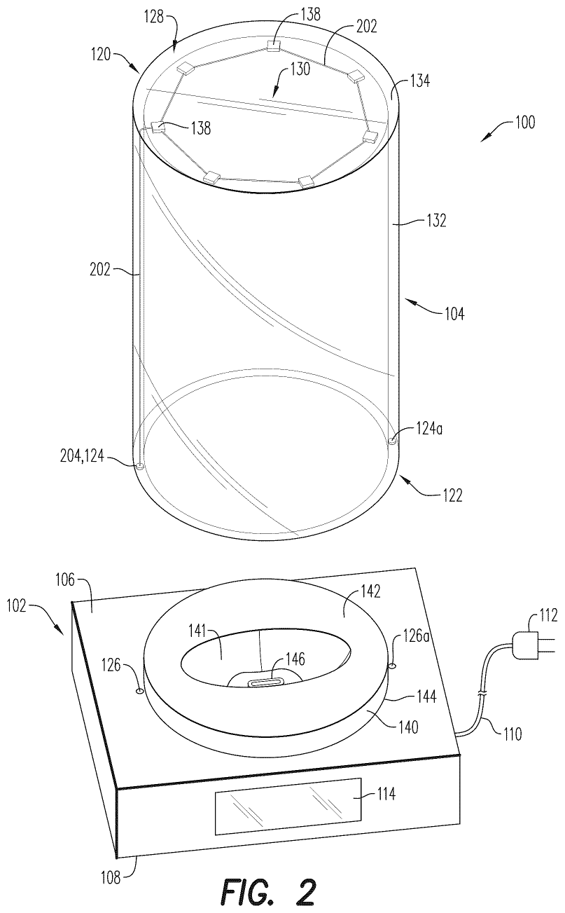

Referring to FIG. 2, a top right, front exploded view of the sterilization system 100 is shown. The sterilization device 104 is shown decoupled from the charging station 102. In some embodiments, the sterilization device 104 may have a circular first end 120. In other embodiments, the first end 120 of the sterilization device 104 may be a different shape such as a square or a rectangle or another shape which is configured such that the enclosure 132 extends from the first end 120 towards the second end 122 to couple with the charging station 102. When the sterilization device 104 is not coupled to the charging station 102, the base magnet 126 and the base magnet 126a may be visible. In some embodiments, the base magnet 126 and the base magnet 126a may be coupled to the first surface 106 of the charging station 102 by an adhesive, a weld, or another suitable means of coupling. In other embodiments, the base magnet 126 and the base magnet 126a may be disposed within the charging station 102 and may not be visible when the sterilization device 104 is removed from the charging station 102.

As shown in FIG. 2, the at least one UV diode 138 may include the plurality of UV diodes 138 disposed on the second surface 136 of the first end 120 of the sterilization device 104. The plurality of UV diodes 138 may be coupled by a wire 202 in some embodiments. The wire 202 may be connected to each of the plurality of UV diodes 138. The wire 202 may extend from the plurality of UV diodes 138 to the second end 122 of the sterilization device 104. In some embodiments, the wire 202 may have a communication point or a contact point 204 that may be positioned at or near the magnet 124. The wire 202 may be flat and thin to minimize its profile within the sterilization device 104. In some embodiments, the UV diodes 138 may have a voltage of about 6.5 volts to 7.2 volts with a current of about 5 milliamps to about 30 milliamps. The wire 202 may have a wire gauge of about 37 American Wire Gauge (AWG) to about 38 AWG to operate the UV diodes 138 as intended. In some embodiments, the magnet 124 may be the contact point 204. The contact point 204 may communicatively couple the plurality of UV diodes 138 to the charging station 102 which may supply power to the plurality of UV diodes 138. In some embodiments, the sterilization device 104 may be communicatively coupled with the charging station 102 via the magnet 124 and the base magnet 126. In other embodiments, the plurality of UV diodes 138 may be communicatively coupled to the contact point 204 via a wireless connection or may be communicatively coupled with the charging station 102 via a wireless connection.

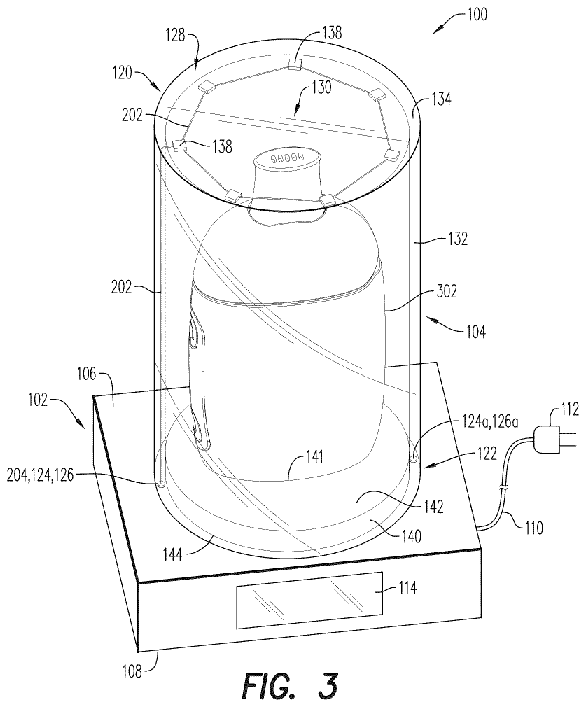

Referring to FIG. 3, the sterilization system 100 is shown with an aerosol-generating device 302 coupled to the charging station 102. The aerosol-generating device 302 may be received by the recess 141 of the dock 140. The aerosol-generating device 302 may be enclosed within the sterilization system 100 such that the enclosure 132 surrounds the aerosol-generating device 302.

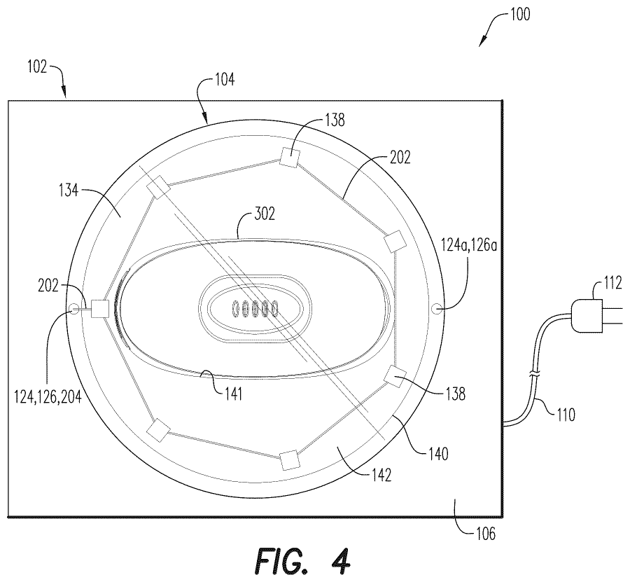

Referring to FIG. 4, a top-down view of the sterilization system 100 including the aerosol-generating device 302 is shown. The aerosol-generating device 302 may be surrounded by the plurality of UV diodes 138 such that UV light from the plurality of UV diodes 138 may contact an exterior of the aerosol-generating device 302 when the plurality of UV diodes 138 are actuated.

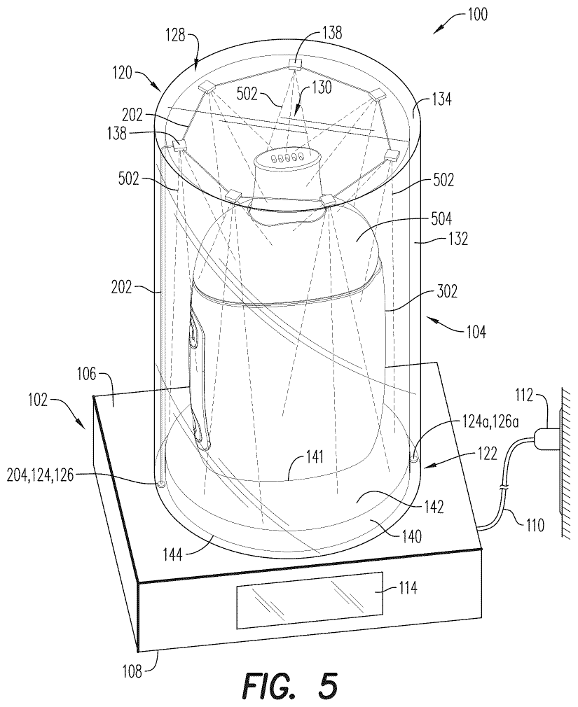

Referring to FIG. 5, the sterilization system 100 is shown including the aerosol-generating device 302 with the sterilization system 100 in operation. When the sterilization system is in operation, each of the plurality of UV diodes 138 may emit UV light 502. The UV light 502 diodes may contact an entire exterior of the aerosol-generating device 302 when the aerosol-generating device 302 is coupled to the charging station 102. As described above, the UV light 502 may be light in a wavelength in the range of 100 nanometers to 280 nanometers. The UV light 502 may be configured to sterilize an exterior 504 of the aerosol-generating device 302 by neutralizing or eliminating any bacteria, mold, viruses, or other potentially harmful contaminants that may be located on the exterior of the aerosol-generating device 302.

The plurality of UV diodes 138 may be configured to actuate when the aerosol-generating device 302 is coupled to the charging station 102. In some embodiments, the plurality of UV diodes 138 may be configured to emit the UV light 502 for a predetermined or a desired amount of time that may be an amount of time to sanitize the exterior of the aerosol-generating device 302. In some embodiments, the communication screen 114 may be configured to output information related to charging or sterilizing the aerosol-generating device 302. For example, the communication screen 114 may output an amount of time that it may take to sterilize the exterior 504 of the aerosol-generating device 302. In some embodiments, the plurality of UV diodes 138 may be configured to turn off if the sterilization device 104 is removed from the charging station 102.

Referring to FIG. 6, a block diagram illustrating example electrical connections among various components of the charging station 102 of the sterilization system 100 is shown. The charging station 102 may include a controller 602 that may be connected to a power source 604, the connection point 146, the communication screen 114, and the contact point 204. The controller 602 may include a memory, processing circuitry (e.g., a processor or other integrated circuitry) and a transceiver. The power source 604 may provide power to the controller 602 for operation thereof and the controller 602 may send commands and/or receive data and information from one or more of the connection point 146, the communication screen 114, and the contact point 204.

In some embodiments, the power source 604 may be included in the charging station 102 and may be any known, or to be developed, battery capable of powering the charging station 102, charging an electronic vaping device inserted into the charging station 102 and/or being charged when the charging station 102 is connected to an external power source.

The connection point 146 may be positioned within the recess 141 of the dock 140 and may be configured to engage a port of an aerosol-generating device to couple the aerosol-generating device to the charging station 102. In some embodiments, the connection point 146 may detect a physical connection between an aerosol-generating device and the connection point 146. The connection point 146 may facilitate transfer of power from the power source 604 to a battery section of the aerosol-generating device. In some embodiments, the connection point 146 may include processing circuitry and at least one resistive element (collectively referred to as circuitry). In one example embodiment, the circuitry may be utilized to generate a resistance when the aerosol-generating device is inserted in, and a physical connection is established with, the connection point 146. In at least one example embodiment, the circuitry may be further configured to send the generated resistance to the controller 602 to indicate the established connection, and the controller 602 may detect the established physical connection between the aerosol-generating device and the corresponding the connection point 146 based on the generated resistance.

In some embodiments, the power source 604 may be coupled to the connection point 146 via a switch 606. Although not specifically shown in FIG. 6, a diode may be connected between the power source 604 and the switch 606 to ensure unidirectional flow of electrical charge from the power source 604 to the battery of the aerosol-generating device through the connection point 146 (e.g., for purposes of charging the battery of the aerosol-generating device).

The charging station 102 may further include a charger input 608 that may be connected to the power source 604 via a switch 612. In this example embodiment, the controller 602 controls the switch 612 to selectively allow power to flow from the charger input 608 to the power source 604. In one example embodiment, the controller 602 closes the switch 612 to allow power to flow from the charger input 608 to the power source 604.

As also shown in FIG. 6, the charger input 608 is connected to the connection point 146 via the switch 606. In this example embodiment, the controller 602 controls the switch 606 to selectively allow power to flow from the power source 604 and/or an external power source to the connection point 146. In one example embodiment, the controller 602 may close the switch 606 to allow power to flow from the power source 604 and/or the external power source to the connection point 146. In another example, the controller 602 may control the switch 606 to allow power to flow from the power source 604 or the charger input 608 to the connection point 146.

As also shown in FIG. 6, the controller 602 may be connected to the communication screen 114 and the contact point 204. The controller 602 may be configured to control the display of the communication screen 114 to output information about the sterilization system 100. The controller 602 may also be connected to the contact point 204 to provide power from the power source 604 to the plurality of UV diodes 138 and to control the operation of the plurality of UV diodes 138 as described above.

According to at least some example embodiments, the switch 612 and the switch 606 may be any well-known switch, including any suitable transistor or other electrical circuit or electromechanical structure.

As shown in FIG. 7, the controller 602 includes processing circuitry 702 (e.g., at least one processor), a memory 704, and a transceiver 706. The processing circuitry 702, the memory 704, and the transceiver 706 are communicatively coupled with one another.

The transceiver 706 may be any known, or to be developed, transceiver for transmission and/or reception of data between the charging station 102 and a remote device. In one example embodiment, the transceiver 706 may enable the establishment of wireless communication between the charging station 102 and the remote device, as will be described below.

In at least one example embodiment, the processing circuitry 702 may include at least one processor. In this example, the processor may be any known, or to be developed, processor configured to execute computer-readable instructions stored on the memory 704. Execution of the computer-readable instructions stored on the memory 704 transforms the at least one processor into a special purpose processor for carrying out the functionality described herein. In some embodiments, the processing circuitry 702 may send appropriate signals/commands to other components of the charging station 102.

Although discussed in some cases with regard to a processor and a memory, according to at least some example embodiments, the controller 602 (or control circuitry or processing circuitry) may be (or include) hardware, firmware, hardware executing software, or any combination thereof. For example, the controller 602 may include one or more Central Processing Units (CPUs), digital signal processors (DSPs), application-specific-integrated-circuits (ASICs), field programmable gate arrays (FPGAs), or other circuitry configured as special purpose machines to perform the functions of the controller 602.

Also described herein is a method of sanitizing the exterior 504 of the aerosol-generating device 302. In some example embodiments, the method can include coupling the aerosol-generating device 302 to the charging station 102, coupling the sterilization device 104 to the charging station 102 such that the sterilization device 104 surrounds the aerosol-generating device 302, actuating the at least one UV diode 138 of the sterilization device 104, and operating the at least one UV diode 138 for a predetermined or a desired amount of time.

In some example embodiments, the predetermined amount of time can be an amount of time to sanitize the exterior 504 of the aerosol-generating device 302. In some example embodiments, the at least one UV diode 138 can be turned off if the sterilization device 104 is decoupled from the charging station 102. In some example embodiments, the method can further include outputting a sanitization status and a charging status on the communication screen 114 of the charging station 102. In some example embodiments, actuating the at least one UV diode 138 of the sterilization device 104 can include directing the UV light 502 from the at least one UV diode 138 to the exterior 504 of the aerosol-generating device 302 to sanitize the exterior 504 of the aerosol-generating device 302.

The systems, apparatuses, and methods described herein may provide significant advantages. For example, the sterilization system 100 may enable the exterior 504 of the aerosol-generating device 302 to be sterilized to help to eliminate adult consumer interaction with bacteria, mold, fungi, viruses, or other harmful contaminates that could be introduced onto the exterior 504 of the aerosol-generating device 302. Sterilization may occur when the aerosol-generating device 302 is coupled to the charging station 102 so the aerosol-generating device is in a non-use mode. The sterilization system 100 may enable the aerosol-generating device 302 to remain cleaner. This may improve an adult consumer's overall satisfaction with the aerosol-generating device 302 because the exterior 504 of the aerosol-generating device 302 may be routinely sterilized without any work from the adult consumer.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

What is claimed is:

1. A sterilization system for an aerosol-generating device, the sterilization system comprising:
a charging station configured to receive the aerosol-generating device; and
a sterilization device comprising at least one UV diode, the sterilization device configured to couple to the charging station and surround the aerosol-generating device when the aerosol-generating device has been received by the charging station, the at least one UV diode configured to sterilize an exterior of the aerosol-generating device when the at least one UV diode is actuated.

2. The sterilization system of claim 1, wherein the sterilization device comprises:
a first end with a central portion and a periphery surrounding the central portion; and
an enclosure extending from the periphery of the first end, the enclosure configured to couple to the charging station.

3. The sterilization system of claim 2, wherein the first end of the sterilization device comprises a first surface with the at least one UV diode and a second surface opposite the first surface.

4. The sterilization system of claim 3, wherein the at least one UV diode is directed away from the second surface of the sterilization device.

5. The sterilization system of claim 3, wherein the at least one UV diode comprises a plurality of UV diodes arranged in a circle on the first surface of the first end of the sterilization device.

6. The sterilization system of claim 3, wherein the at least one UV diode comprises a plurality of UV diodes arranged to direct UV light to the exterior of the aerosol-generating device.

7. The sterilization system of claim 3, wherein the at least one UV diode comprises a plurality of UV diodes on the first surface of the first end of the sterilization device directed towards the charging station such that UV light from the plurality of UV diodes contacts an entire exterior of the aerosol-generating device when the aerosol-generating device is coupled to the charging station.

8. The sterilization system of claim 2, wherein the enclosure comprises at least one magnet configured to couple to at least one magnet of the charging station.

9. The sterilization system of claim 8, wherein the at least one magnet of the enclosure and the at least one magnet of the charging station comprise neodymium magnets.

10. The sterilization system of claim 1, wherein the at least one UV diode is actuated when the aerosol-generating device is received by the charging station and when the sterilization device is coupled to the charging station.

11. The sterilization system of claim 1, wherein the at least one UV diode is turned off if the sterilization device is decoupled from the charging station.

12. The sterilization system of claim 1, wherein the charging station comprises a communication screen.

13. The sterilization system of claim 12, wherein the communication screen is configured to output information related to a charge status of the aerosol-generating device.

14. The sterilization system of claim 12, wherein the communication screen is configured to output information related to a sterilization status of the aerosol-generating device.

15. The sterilization system of claim 1, wherein the sterilization device is communicatively coupled to the charging station.

16. The sterilization system of claim 15, wherein the sterilization device is communicatively coupled to the charging station at a contact point, the contact point configured to be communicatively coupled to the at least one UV diode.

17. The sterilization system of claim 1, wherein the sterilization device comprises a plastic UV filter.

18. The sterilization system of claim 1, wherein the at least one UV diode comprises a UV-C diode.

19. The sterilization system of claim 1, wherein the at least one UV diode comprises a UV-C light ring diode.

20. The sterilization system of claim 1, wherein the at least one UV diode is configured to emit UV light in a wavelength range of 100 nanometers to 280 nanometers.
